(19) United States
(12) Patent Application Publication
(10) Pub. No.: US 2026/0224891 A1
(43) Pub. Date: Aug. 6, 2026

(54) DEEP BRAIN HIGH CHANNEL COUNT NEURO STIMULATION AND MONITORING ARRAY

(71) Applicant: The Regents of the University of California, Oakland, CA (US)

(72) Inventors: Hoi Sang U, Rancho Santa Fe, CA (US); Shadi A. Dayeh, San Diego, CA (US)

(21) Appl. No.: 19/151,825

(22) PCT Filed: Jan. 31, 2024
(86) PCT No.: PCT/US2024/013695

§ 371 Date: Jul. 29, 2025

Related U.S. Application Data
(60) Provisional application No. 63/482,935, filed on Feb. 2, 2023.

(51) Int. Cl. A61N 1/05; A61N 1/36

(52) U.S. Cl. CPC A61N 1/0534; A61N 1/36128

(57) ABSTRACT

A microelectrode grid array for neuromonitoring from brain cavities includes a mesh of interweaved microscale medical grade wire columns and rows. There is biocompatible insulation of the mesh. Exposed contact regions on the wire columns and rows form a plurality of electrodes. Electrical wires connected from the mesh are connectable away from the mesh to an electrophysiological recording and stimulation system. The mesh is foldable into a delivery shape to fit into and be delivered by a ventricular catheter and expandable to a deployed shape upon deployment through a distal end of the ventricular catheter to conform to the shape of a region of the brain with the exposed contact regions contacting an ependymal lining of the region of the brain. WO

### PRIORITY CLAIM AND REFERENCE TO RELATED APPLICATION

The application claims priority under 35 U.S.C. § 119 and all applicable statutes and treaties from prior U.S. provisional application Ser. No. 63/482,935, which was filed Feb. 2, 2023.

### STATEMENT OF GOVERNMENT INTEREST

This invention was made with government support under grant numbers UG3NS123723-01, R01NS123655-01, DP2-EB029757 awarded by the National Institutes of Health. The government has certain rights in this invention.

### FIELD

A field of the invention is brain sensing and stimulation. The invention provides a neuro stimulation and monitoring array that can be delivered via a minimally invasive delivery device. The invention provides for sensing and stimulation of deep brain structures and networks.

### BACKGROUND

The ventricular system is composed of interconnected cavities filled with cerebrospinal fluid within the cerebral hemispheres. These cavities are surrounded by deep brain structures and networks. The ventricular system is a series of cerebral spinal fluid (CSF) filled chambers in the middle of the brain. In the cerebral hemispheres, it is surrounded by a dense network of axonal fibers communicating between different brain regions (e.g., cortical regions to cortical regions or cortical regions to the basal nuclei/hypothalamus/brainstem/spinal cord). The ventricles in turn cap around the nuclei in the middle and base of the brain. It is therefore situated in the center of critical control/relay centers essential for brain function. The ventricular system is lined by a thin layer of ependymal cells.

The central nervous system (CNS) is essential in the control of functions in daily life such as mobility, sensation, sight, speech and hearing. It is also the organ which defines our individual characteristics such as emotion, memory, thought, creativity and motivation. These complex functions entail the coordination of the cerebral cortex and cell groups (nuclei) residing in the middle of the brain.

In addition to centers involved in motor control, other neuronal centers at the base of the cerebral hemispheres are involved in essential functions such as memory. Cholinergic cells in the Basal Nucleus of Meynert project connections to the hippocampus via the fornix and are essential in mediating memory function. Destruction of these cells leads to memory loss. This destruction can be rescued by nerve growth factor (NGF) delivered into the ventricular systems or by the grafting to the Basal Nucleus of Meynert of cells genetically engineered to produce NGF. See, Ridley et al, “Restoration of cognitive abilities by cholinergic grafts in cortex of monkeys with lesions of the basal nucleus of Meynert,” Neuroscience, Vol. 63(3) pp 653-66 (2014); Tuszynski, M H et al, “A Phase I Clinical Trial of Nerve Growth Factor Gene Therapy for Alzheimer's Disease,” Nature Medicine, April 2005 (2005); Tuszynski, M H et al, “Nerve Growth Factor Gene Therapy: Activation of Neuronal Responses in Alzheimer Disease,” JAMA Neurol. 72(10):1139-47 (2015). The end result is evidenced by some restoration of memory and other higher cortical functions such as conceptualization. Despite these positive outcomes, the neuronal networks mediating these effects are poorly understood in part due to the lack of any device that can monitor coordinating pathways.

The central nervous system (CNS) and the endocrine system are the two main controlling systems for the development and function of the human body. Coordination of their functions is essential for the appropriate and adequate function in human physiology. These interactions occur primarily through communications between nuclei in the base of the brain such as those residing in the hypothalamus. In addition to this neural endocrine interaction, deep brain nuclei are also critical in important brain functions ranging from the fine control of movement directed from the cerebral cortex to the control of complex functions such as emotions and eating which may have endocrine implications/involvements. Many of these functions are poorly understood due partly to the lack of a safe tool to monitor their activities.

Electrophysiological monitoring of cortical activities has instead been mainly limited to the placement of electrodes on the scalp or directly on the cerebral cortex. Modulations of these electrophysiological activities have included stimulation of specific regions of interest. The results of these electrophysiological modulations are recorded/observed in specific end organs via end-organ specific devices such as the use of electromyography (EMG) or other clinical measures applied to the end-organs.

The mechanisms involving fiber circuits/networks responsible for modulating influences between the cerebral cortex and the end-organ is not well understood due to an inability to directly record activities of the intervening networks/pathways. An understanding of such intervening influences is also complicated by the participation of nuclei at the interior/base of the brain such as the basal ganglia and the thalamus. Even though individual nuclei can be monitored and manipulated by depth electrodes, they target only a limited number of selective nuclei and not an entire network.

Current methods employed in the monitoring and modulation of deep brain structures involved in motor control entail the insertion of devices such as a cylindrical electrode to the target for recording, lesioning or stimulation (DBS). Henderson, J., “Connectomic Surgery: Diffusion Tensor Imaging (DTI) Tractography As A Targeting Modality For Surgical Modulation Of Neural Networks,” Frontiers in Intergrative Neuroscience 6:1-6 (2012); Lozano et al., “Deep Brain Stimulation: Current Challenges And Future Directions,” Nat Rev 15 (3):148-160 (2019); Kraus et al., Technology Of Deep Brain Stimulation: Current Status And Future Directions,” Nature Rev 17:75-87 (2021); Aum & Tierney, “Deep Brain Stimulation: Foundations And Future Trends Frontiers In Bioscience,” Landmark, 23:162-182 (2018).

A typical cylindrical electrode is about 1 mm in diameter and can have as many as 16 electrical contacts, each with a length of at least 1.5 mm or more. The cylindrical electrode can reveal activities of a specific target nucleus but not its affluent or effluent pathways. It can also under sample a volume of the target region due to poor spatial resolution. Using multiple electrodes could provide more resolution but is not practical or safe. Thus, the current methods are frequently restricted to one region of the brain, e.g., EEG of specific cortical regions or deep brain electrodes inserted into specific basal nuclei.

Monitoring and modulation of cortical functions has been conducted by implantation of microelectrodes in specific regions of the cerebral cortex. Studies with such electrodes have identified complex cortical control mechanisms such as that in speech. See, e.g., Wilson G. H. et al., “Decoding Spoken English from Intracortical Electrode Arrays in Dorsal Precentral Gyrus,. J Neural Eng 17(6), (2020); Hosman T, et al. “Auditory Cues Reveal Intended Movement Information In Middle Frontal Gyrus Neuronal Ensemble Activity Of A Person With Tetraplegia,” Scientific Reports, 11(98). (2021). Individual detailed cortical function study does not reveal mechanism(s) involved in comprehensive brain function.

### SUMMARY OF THE INVENTION

A preferred embodiment provides a microelectrode grid array for interoperative neuromonitoring includes a mesh of interweaved microscale medical grade wire columns and rows. There is biocompatible insulation of the mesh. Exposed contact regions on the wire columns and rows form a plurality of electrodes. Electrical wires connected from the mesh are connectable away from the mesh to an electrophysiological recording and stimulation system. The mesh is foldable into a delivery shape to fit into and be delivered by a ventricular catheter and expandable to a deployed shape upon deployment through a distal end of the ventricular catheter to conform to the shape of a region of the brain with the exposed contact regions contacting an ependymal lining of the region of the brain.

### BRIEF DESCRIPTION OF THE DRAWINGS

FIGS. 1A-1D show a preferred embodiment microelectrode grid array;

FIGS. 2A-2C show the microelectrode grid array being deployed on the trigone of the lateral ventricle and a preferred balloon catheter delivery system; and

FIGS. 3A-3C show another preferred embodiment microelectrode grid array and a preferred method of fabrication.

### DETAILED DESCRIPTION OF THE PREFERRED EMBODIMENTS

Preferred embodiments provide a microelectrode grid array for interoperative neuromonitoring configured to introduced via a minimally invasive delivery device into the ventricular system to monitor and manipulate deep brain structures/networks. A preferred microelectrode grid array is configured to be introduced into the ventricular system and sized and configured to rest on and cover the ependymal lining of the ventricles and the underlying deep brain structures/networks. This provides close contact with important deep brain nuclei, as well as with the relay pathways. So positioned, both deep brain nuclei and the periventricular fiber tracts can be monitored and modulated by the microelectrode grid array.

Preferred microelectrode grid arrays and delivery devices permit the simultaneous modulation and monitoring of multiple brain regions and their interacting pathways. This provides a powerful tool for more comprehensive study of brain functions, and for tailoring interventions to disease treatments. An array of the invention delivered via a delivery device is believed to be the first of its kind that can rest on the surface of the ventricles within the brain structure to modulate brain activity directly at relevant regions.

Preferred microelectrode grid arrays can be delivered via a delivery device into close contact with important deep brain nuclei and with associated relay pathways. The delivery device and array are dimensioned and configured to be placed in that position. Both deep brain nuclei and the periventricular fiber tracts can be monitored and modulated by electrodes of the array. Preferred microelectrode grid arrays permit monitoring and modulation of deep brain structures for recording, lesioning or stimulation.

Preferred embodiments provide patient-specific self-expandable electrophysiological meshes (SEEM) that can be placed with minimal invasiveness in the cerebral ventricles of large animals including humans and are configured to expand and latch to the ventricle sidewalls to understand and modulate the interactions between the brain and endocrine systems.

Preferred embodiments include a microelectrode grid array for interoperative neuromonitoring includes a mesh of interweaved microscale medical grade wire columns and rows. There is biocompatible insulation of the mesh. Exposed contact regions on the wire columns and rows form a plurality of electrodes. Electrical wires connected from the mesh are connectable away from the mesh to an electrophysiological recording and stimulation system. The mesh is foldable into a delivery shape to fit into and be delivered by a ventricular catheter and expandable to a deployed shape upon deployment through a distal end of the ventricular catheter to conform to the shape of a region of the brain with the exposed contact regions contacting an ependymal lining of the region of the brain. The medical grade wire columns and rows can be made of titanium. Wire bundles from the rows and columns are preferably disposed along the boundaries of the mesh. The bundles can extend proximally to exit a proximal end of the ventricular catheter.

There can be boundary stylets at boundaries of the mesh and a central stylet connectable to a distal end of the mesh. Axial proximal movement of the central stylet causes radial expansion of the boundary stylets and the mesh to the deployed shaped and axial distal movement of the central stylet causes radial contraction of the boundary stylets and folding of the mesh to the delivery shape. A distal end of the central stylet and the distal end of the mesh can include a threaded connection to each other. The central stylet can be stiffer than the boundary stylets.

Boundary stylets can be made of shape memory material expands radially when released from the ventricular catheter to expand the mesh to the deployed shaped.

The mesh can be in the form of a 3D shape with neutral stress configured to be axially stretchable to radially retracted position sized to fit within a ventricular catheter. The 3D shape can be an egg-like shape at its outer boundaries. The mesh can be sized and shaped to conform to the ependymal lining of third ventricle or to conform to the ependymal lining of the trigone of the lateral ventricle.

The mesh can be mounted upon a self-expanding polydioxanone mesh.

A ventricular catheter can include any microelectrode grid array within a lumen of the catheter. The catheter can include an inner and outer catheter, wherein a proximal portion of the mesh is connected to a distal portion of the inner catheter. The mesh can be mounted upon an inserter, the inserter being within the inner catheter.

Preferred embodiments of the invention will now be discussed with respect to experiments and drawings. Broader aspects of the invention will be understood by artisans in view of the general knowledge in the art and the description of the experiments that follows.

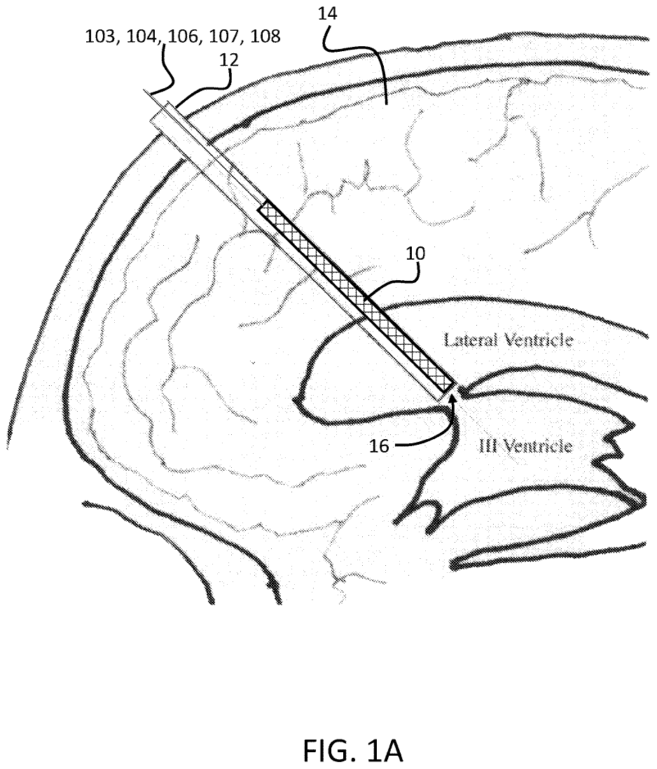

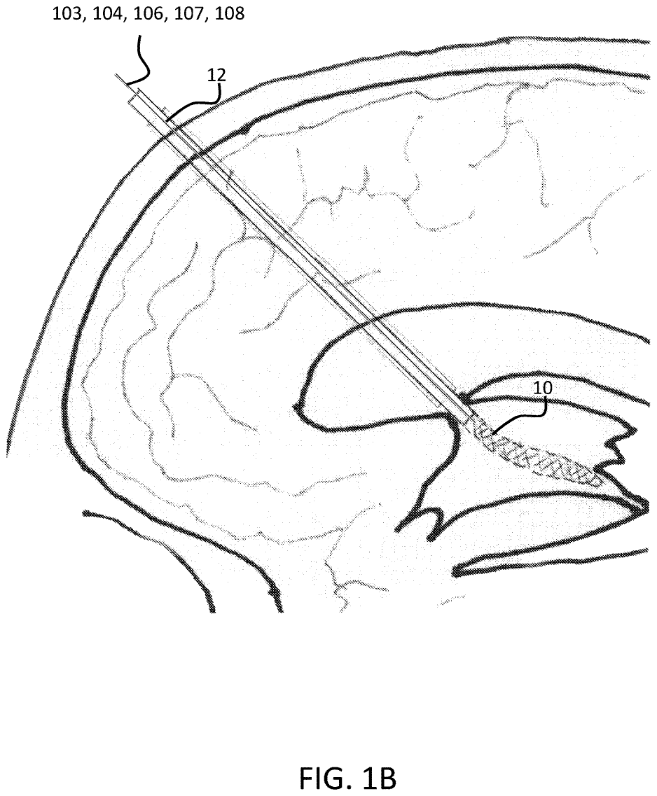

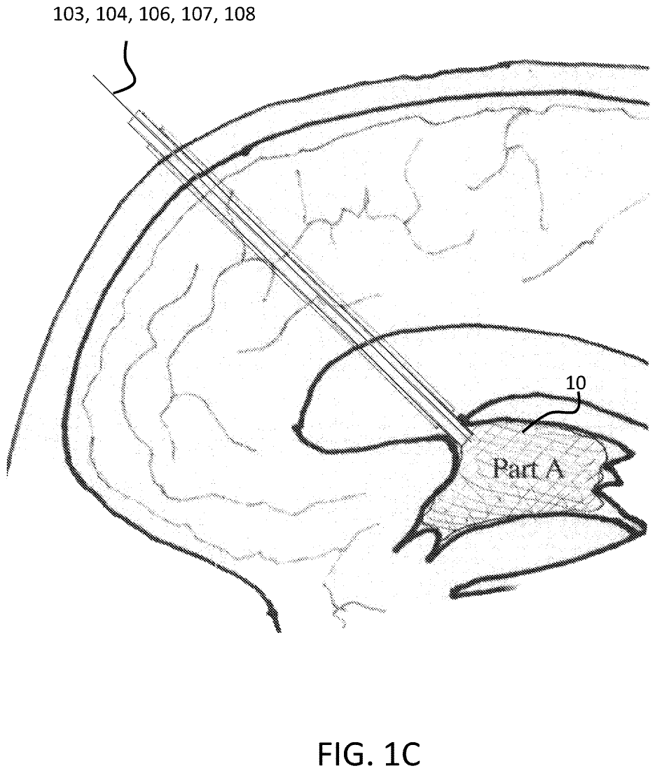

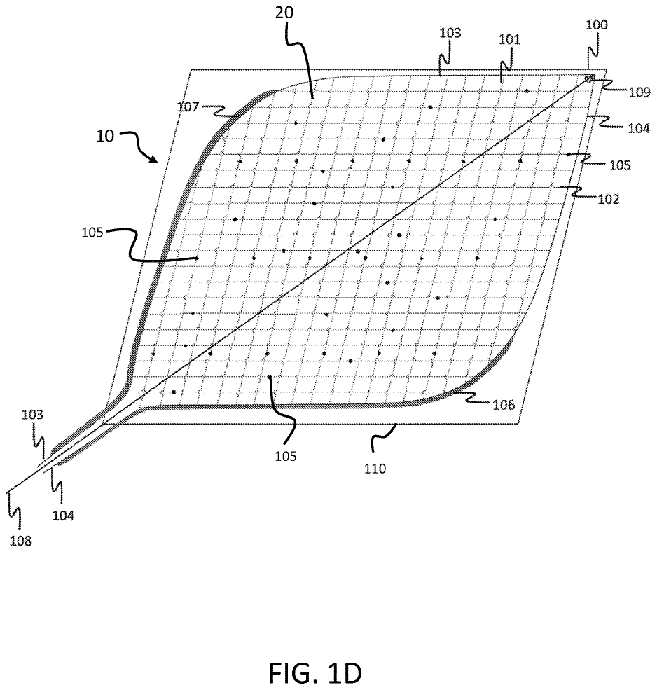

FIGS. 1A-1D show a preferred embodiment microelectrode grid array 10 and its delivery device 12. The delivery device 12 is a catheter configured to access ventricles of the brain 14. The delivery device is configured in the same manner as ventricular catheters used to drain cerebrospinal fluids (CSF) such as the VentriClear™ offered by Medtronic or the CerebroFlor® offered by Integra. The ventricular catheter 12 holds microelectrode grid array 10 in a collapsed form to deliver it through a distal end 16 of the ventricular catheter 12. In its collapsed form, the microelectrode grid array 10 carried into the ventricular system of the brain by the ventricular catheter 12.

The microelectrode grid array 10 (see FIG. 1D) is an interwoven electrode structure 20, with a pattern that is similar to a vascular stent that can be expanded and contracted upon its deployment and retrieval. The microelectrode grid array 10 can be expanded by a balloon, for example, or can be self-expanding. The microelectrode grid array 10 expands into a three-dimensional shape upon deployment. The size of the microelectrode grid array 10 can be in the range of 1 mm³ to 40 cm³ for deep brain deployment.

The expandable microelectrode grid array 10 includes a mesh of interweaved conductive wire columns 101 and rows 102 and is foldable into a delivery shape (FIGS. 1A and 1B) to fit into a lumen of and be delivered by a ventricular catheter and expandable to a deployed shape (FIG. 1D shows a two-dimensional top view when the microelectrode grid array 10 is spread out on a temporary substrate 110 and will expand to a balloon-like deployed shape when released from the substrate) upon deployment through a distal end of the ventricular catheter to conform to the shape of a region of the brain. The columns 101 and rows 102 are individually addressable, i.e. the columns 101 are insulated from each other and from the rows 102, and the rows 102 are insulated from each other and from the columns. A preferred conductive wire is made of platinum, but other biocompatible metals can be used, including alloys of platinum/iridium, platinum/tungsten, nickel/chromium, and the MP35N Alloy. These conductive wire columns 101 and rows 102 are medical grade, Individually electrically insulated with an insulator, e.g., a flexible polymer, such as an aromatic polyimide resin. Other suitable insulators include thin ceramic, polyurethane, polyurethane/nylon, polyimide, polyester, polyesterimide, and PTFE. Wires have been fabricated to 18 microns in diameter but can be made smaller to accommodate more sensing sites in the microelectrode grid array 10. The wires can also be made larger if fewer sensing sites are needed, e.g. 1 mm, however the wires must be small enough such that the mesh fits within the catheter for delivery by the catheter.

At the boundaries of the platinum wire mesh 20 are thin metal, e.g. stainless-steel, boundary stylets 103, 104 for columns 101 and rows 102, respectively. The boundary stylets 103 and 104 are configured as structural features and help to contract and expand the microelectrode grid array 10 based upon movement of a central stylet 108. The wires from the columns 101 are bundled along boundaries of the mesh 20 into 106 bundle next to the boundary stylet 104, and the wires from the rows 102 are bundled along boundaries of the mesh 20 into a bundle 107 along to the boundary stylet 103.

The entire mesh 20 is temporally supported by a substrate 110 during fabrication. A preferred fabrication uses thin parylene C (2-4 microns coated on a glass substrate or equivalent substrate (e.g., silicon). At the end of fabrication, most of the parylene film is etched out to leave the mesh 20, which can then be peeled off the substrate. A plurality of exposed contact regions 105 can be formed, for example, by laser engraving or top-down lithography processes. The exposed contact regions 105 are distributed throughout the mesh 20 to provide a plurality of stimulation and sensing locations through the mesh. Exposed contact regions 105 can number a few up to a thousand or more, depending upon the thickness of the wires in the mesh and the diameter of the delivery catheter. A thicker metal (e.g. stainless steel) central stylet 108 is guided through the mesh and fixed in a notch 109 toward the distal corner of the wire mesh 102. One manner of fixing is threading, e.g. the central stylet 108 preferably has a male thread at its distal end and the threaded notch a female thread. In general, the central stylet 108 is fixed 109 to enable mechanical movements for expanding and contracting the mesh 20 without separating from the notch 109.

The central stylet 108 moves axially with respect to stylets 103 and 104 in order to contract or expand the microelectrode array 10. For example, when boundary stylets 103 and 104 are fixed in position and the central stylet 108 is distally advanced with respect to boundary stylets 103 and 104, the wire mesh 20 will contract into a narrow body that can be inserted into the catheter 12. When the central stylet 108 is retracted backwards with regards to 103 and 104, the wire mesh 20 will expand to fill the space it is enclosed in. The surface tension together with stainless steel stylets 103 and 104 will hold the mesh in its expanded position. Wire bundles 106 and 107 can be connected to an electrophysiological recording and stimulation system.

The exposed contact regions 105 serve as monitoring and modulating electrodes when the microelectrode grid array 10 is deployed so that the electrodes rest gently on the ependymal lining of the ventricles. While stylets 103, 104, and central stylet 108 are a preferred way to expand and deploy the microelectrode grid array 10, another option includes self-expanding shape memory frame material, e.g. nitinol used as the stylets 103 and 104. In that case, the stylets 103 and 104 need not be accessible through the proximal end of the catheter 12. Instead, the shape memory material of the stylets 103 and 104 will expand the microelectrode grid array 10 as it is advanced via the central stylet 108 or an inner catheter until it emerges from a distal end of the catheter 12 and expands. The curved outer surfaces of the proximal portion of the microelectrode grid array 10 permit retraction into the catheter 12 and serve to smoothly fold the microgrid array 10 back into its folded/elongated position that fits within the inner diameter of the catheter 12.

The overall shape of the microelectrode grid array 10 in a two-dimensional plane through the array 10 is similar to a rose leaf, generally an elongated oval that narrows to the distal end at the notch 109. This shape is preferred to latch on the inner surfaces of three dimensional brain cavities, and to collapse/contract into a small form factor for extraction with damaging the delicate tissue.

FIGS. 1A-1B show the microelectrode grid array 10 inserted via an anterior approach to conform to the ependymal lining of third ventricle via anterior lateral ventricle entry. The catheter 12 is sized to be inserted through the narrow Foramen Munro to access the third ventricle. In FIGS. 1A-1B the microelectrode grid array 10 is sized and shaped to conform to the ependymal lining of third ventricle. Other deployments are possible. For example, the catheter 12 can access the trigone of the lateral ventricle and the microelectrode grid array 10 can be deployed there. It is a posterior approach, and the microelectrode grid array is shaped and sized to conform to the ependymal lining of the trigone of the lateral ventricle.

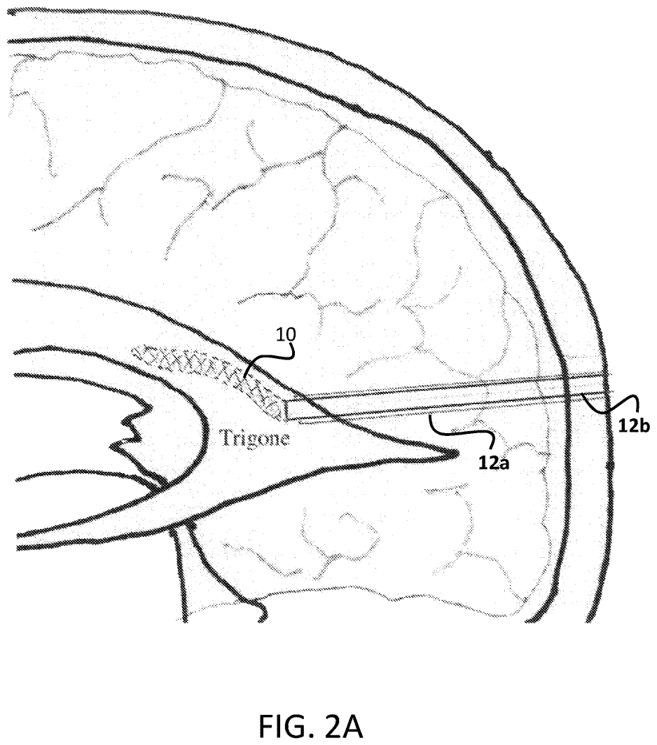

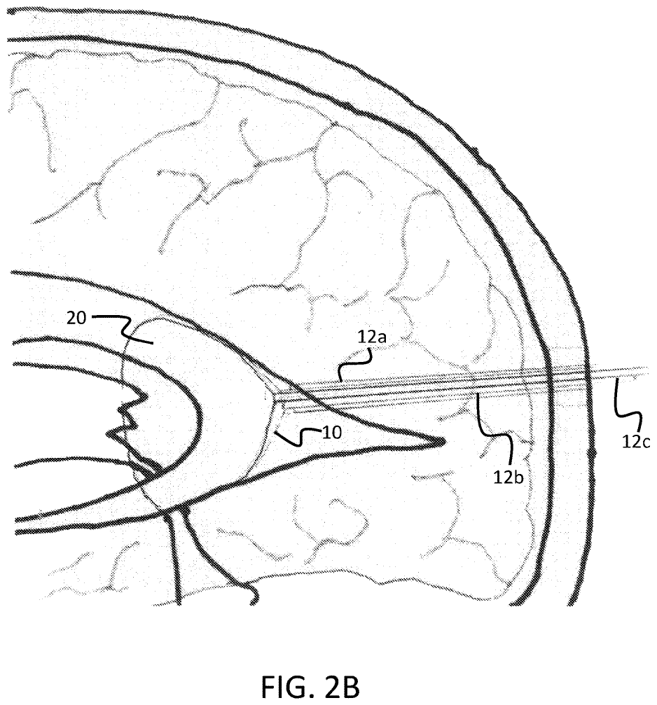

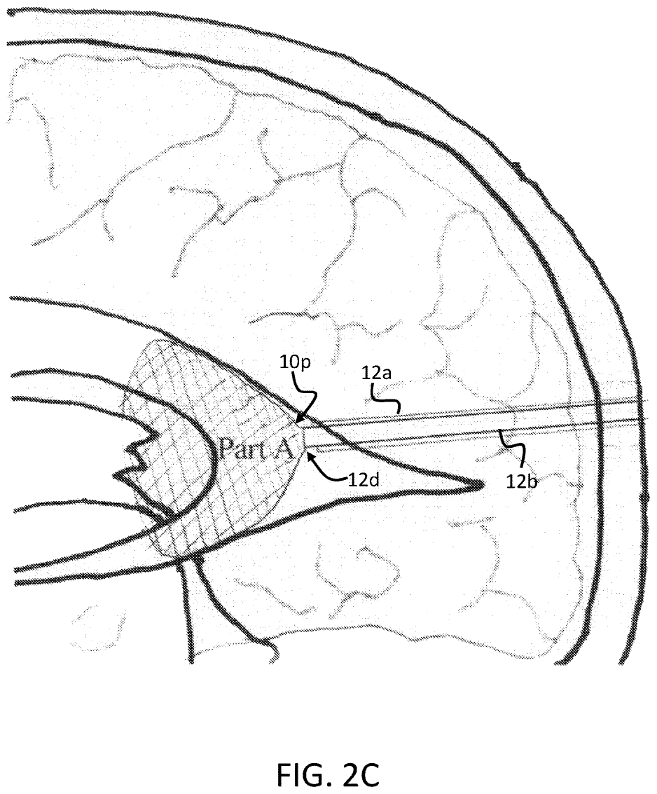

FIGS. 2A-2C show the microelectrode grid array 10 being deployed on the trigone of the lateral ventricle and a different deployment strategy. A catheter for deployment includes an outer 12a, inner catheter 12b and a microelectrode inserter 12c (FIG. 2B) Prior to deployment, the microelectrode grid array is held on the inserter 12c and compressed by the inner catheter 12b. When released from the inner catheter 12b the microelectrode grid array 10 expands like a balloon, into contact with the targeted organ regions. In this instance, the microelectrode grid array 10 need not include any stylets, as it is configured to expand like a balloon when it is released from the inner catheter 12b. After the microelectrode grid array 10 is deployed (FIG. 2C), the inserter 12c can be withdrawn. A proximal end 10p of the microelectrode grid array 10 is connected to a distal end 12d of the inner catheter 12b, which permits it to be retracted into the outer catheter 12a. Wire bundles 106 and 107 are omitted for simplicity of illustration but extend out through a proximal end of the inner catheter 12b as shown in FIGS. 1A-1C.

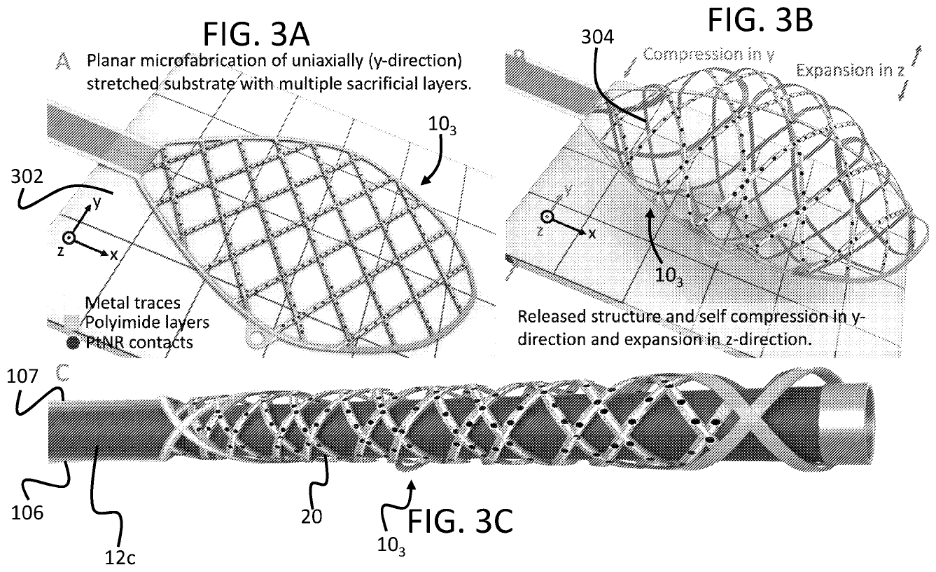

FIGS. 3A-3C show another preferred microelectrode grid array 103 and a preferred fabrication process. FIGS. 3A and 3B show it fabrication, and FIG. 3C show the mesh 20 of the array mounted on the inserter, and the microfabricated wire trace bundles 106 and 107 extending distally. The mesh includes individually insulated rows and columns as in the embodiments above, and exposed contact regions.

In FIG. 3A the mesh 20 is formed by thin-film microfabrication of the polyimide covered mesh on a stretched substrate 302 that includes multiple sacrificial layers. Substrate 302 is stretched in the x-direction and mounted on a substrate carrier (e.g. glass plate or Si wafer). The substrate 302 can include an elastomer, such as silicone molded into a sheet, and then stretched and adhered to a substrate carrier. The microelectrode grid array 103 is then fabricated on a sacrificial layer, could be a metal, e.g., titanium or a dielectric layer, e.g. silicon dioxide, with anchor points of the grid directly touching the stretched elastomer. Fabrication of the microelectrode grid array 103 is continued until its 3D layered structure is completed. When the sacrificial layers are etched award and the elastomer layers are released from the underlying substrate carrier, microelectrode grid array 103 releases into its intended shape shown in FIG. 3B. Specifically, the mesh 20 wire materials on top of stretched layers contract in plane with the silicone and expand out of plane to have a neutral stress position. The elastomer is then separated from the grid array 10₃. No stylets are included, as the neutral position of the mesh 20 will be re-established after the mesh is released from a catheter.

Specifically, as the stretched substrate 302 is released, it contracts in the x-direction—to minimize its internal stresses—and mesh 20 on top also contract in the x-direction. To balance the in-plane contractions of mesh 20, and given that it consists of free standing mesh components, mesh 20 expands in the out-of place z-direction to form its three-dimensional shape shown in FIG. 2B, which can be an egg-like shape at its outer boundaries as shown in FIG. 2B. The mesh 20 can be axially expanded causing radial retraction and mounted on the inserter 12c as shown in FIG. 3.

An alternative to the fabrication of mesh 20 on stretchable substrate 302 is the fabrication of mesh 20 on a normal substrate and the release of mesh 20 without any internal stresses from the normal substrate. If mesh 20 is then attached by the aid of polymer layers to a stretched substrate 302, the same mechanism of self-expansion as in FIGS. 3A and 3B. For this fabrication, substrate 302 needs to be thin, ideally less than 10 μm, so as not to increase the total thickness of the mesh. Another alternative is the mounting of a mesh 20 on a shape memory material, the mesh 20 can also be formed upon on a self-expandable and resorbable polydioxanone (PDS) mesh 304, like those that are conventionally used for stents. The PDS mesh can be attached to the catheter such that after deployment of the microelectrode grid array 103 in the third ventricle, manipulation of wire trace bundles 106 and 107 of the PDS mesh can contract the microelectrode grid array 103 to explant it without harming tissue.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A microelectrode grid array for interoperative neuromonitoring, comprising:
a mesh of interweaved microscale medical grade wire columns and rows;
biocompatible insulation of the mesh;
exposed contact regions on the wire columns and rows forming a plurality of electrodes; and
electrical wires connected from the mesh and being connectable away from the mesh to an electrophysiological recording and stimulation system, wherein the mesh is foldable into a delivery shape to fit into and be delivered by a ventricular catheter and expandable to a deployed shape upon deployment through a distal end of the ventricular catheter to conform to the shape of a region of the brain with the exposed contact regions contacting an ependymal lining of the region of the brain.

2. The microelectrode grid array of claim 1, comprising:
boundary stylets at boundaries of the mesh;
a central stylet connectable to a distal end of the mesh; wherein axial proximal movement of the central stylet causes radial expansion of the boundary stylets and the mesh to the deployed shaped and axial distal movement of the central stylet causes radial contraction of the boundary stylets and folding of the mesh to the delivery shape.

3. The microelectrode grid array of claim 2, wherein a distal end of the central stylet and the distal end of the mesh comprise a threaded connection to each other.

4. The microelectrode grid array of claim 2, wherein the central stylet is stiffer than the boundary stylets.

5. The microelectrode grid array of claim 2, wherein the boundary stylets are made of shape memory material expands radially when released from the ventricular catheter to expand the mesh to the deployed shaped.

6. The microelectrode grid array of claim 2, wherein the boundary stylets comprise stainless steel.

7. The microelectrode grid array of claim 1, wherein the medical grade wire columns and rows comprise titanium.

8. The microelectrode grid array of claim 1, comprising wire bundles from the wire columns and rows, the wire bundles being disposed along the boundaries of the mesh.

9. The microelectrode grid array of claim 8, wherein the wire bundles extend proximally to exit a proximal end of the ventricular catheter.

10. The microelectrode grid array of claim 1, wherein the mesh comprises a 3D shape with neutral stress configured to be axially stretchable to radially retracted position sized to fit within a ventricular catheter.

11. The microelectrode grid array of claim 10, wherein the 3D shape comprises an egg-like shape at its outer boundaries.

12. The microelectrode grid array of claim 1, wherein the mesh is sized and shaped to conform to the ependymal lining of third ventricle.

13. The microelectrode grid array of claim 10, wherein the mesh is sized and shaped to conform to the ependymal lining of the trigone of the lateral ventricle.

14. The microelectrode grid array of claim 1, wherein the mesh is mounted upon a self-expanding polydioxanone mesh.

15. A ventricular catheter comprising a microelectrode grid array of any of claim 1 in its lumen.

16. The ventricular catheter of claim 15, comprising an inner and outer catheter, wherein a proximal portion of the mesh is connected to a distal portion of the inner catheter.

17. The ventricular catheter of claim 15, comprising wherein the mesh is mounted upon an inserter, the inserter being within the inner catheter.
